(19) United States
(12) Patent Application Publication
(10) Pub. No.: US 2024/0358946 A1
(43) Pub. Date: Oct. 31, 2024

(54) CONTROLLING THE CONCENTRATION OF DEOXYHEMOGLOBIN IN A SUBJECT

(71) Applicant: THORNHILL SCIENTIFIC INC., North York (CA)

(72) Inventors: Joseph Arnold FISHER, Thornhill (CA); James DUFFIN, Toronto (CA); Olivia SOBCZYK, Etobicoke (CA); Ece Su SAYIN, Windsor (CA); David J. MIKULIS, Oakville (CA); Julien POUBLANC, North York (CA); Jacob B. SCHULMAN, Toronto (CA)

(21) Appl. No.: 18/579,302

(22) PCT Filed: Jul. 18, 2022
(86) PCT No.: PCT/IB2022/056603

§ 371 Date: Jan. 12, 2024

Related U.S. Application Data
(60) Provisional application No. 63/222,827, filed on Jul. 16, 2021.

(51) Int. Cl. A61M 16/00; A61M 16/12

(52) U.S. Cl. CPC A61M 16/024; A61M 16/12; A61M 2202/0208; A61M 2202/0225; A61M 2202/0266; A61M 2205/3303; A61M 2205/3334; A61M 2205/3368; A61M 2205/505; A61M 2210/1039; A61M 2230/205

(57) ABSTRACT

The speed and range of the transition to low lung PO₂ can be optimized such that it approaches the PO₂ and timing profile of reoxygenation from a hypoxic profile. However, such hypoxic “spikes” are much more difficult to implement than re-oxygenation. A solution is to control the independent variables which contribute to hypoxia in the lung. These variables may be controlled in alone or in aggregate to minimize the time required to generate a target profile of transient lung hypoxia. In particular, a rapid decrease in [dOHb] can be implemented by breathing deeply, exhaling at least a portion of the functional residual capacity, lowering the PO₂ at baseline, increasing the breathing rate, and increasing the PCO₂ to shift the oxygen-hemoglobin dissociation curve to the right.

### CROSS-REFERENCE TO RELATED APPLICATION(S)

This application claims the benefit of U.S. provisional application entitled METHODS TO IMPROVE THE EASE, SPEED AND ACCURACY OF ATTAINING TARGETED SaO₂, having Ser. No. 63/222,827, filed Jul. 16, 2021 and incorporated by reference herein.

### FIELD

The present specification is directed to perfusion MRI, and specifically dynamic susceptibility contrast (DSC) MRI with deoxyhemoglobin as a contrast agent.

### BACKGROUND

Deoxyhemoglobin has been explored as a safer alternative to gadolinium as contrast agent for magnetic resonance imaging (MRI). Deoxyhemoglobin provides a number of advantages over gadolinium as a contrast agent. Firstly, as an endogenous molecule, it is safe to administer and causes few adverse reactions. Deoxyhemoglobin does not accumulate or recirculate because it reverts to oxyhemoglobin after returning to the lungs. Secondly, inspired gas almost instantaneously distributes throughout the lungs with each inspiration, resulting in near instantaneous equilibration with the pulmonary blood volume. Thirdly, it is safe and well-tolerated to target repeated changes in [dOHb], permitting individuals and populations to be studied over time.

In order to be useful as a contrast, deoxyhemoglobin must be precisely controlled.

### SUMMARY

An aspect of the present disclosure provides a method of inducing a hypoxic bolus of arterial blood in a subject. A sequential gas delivery device is used to target a first end tidal partial pressure of oxygen (P_{ET}O₂) and then a second P_{ET}O₂ which is lower than the first P_{ET}O₂. In order to improve the speed at which the PO₂ in the lung decreases, the first and second P_{ET}O₂ are selected based on an oxygen-hemoglobin dissociation curve. Furthermore, at least one variable that contributes to the rate of change in the partial pressure of oxygen in the lung is controlled while targeting the second P_{ET}O₂. After targeting the P_{ET}O₂, the sequential gas delivery device targets a third P_{ET}O₂ that is higher than the second P_{ET}O₂.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a block diagram of a system for inducing a hypoxic bolus in a subject.

FIG. 2 is a flowchart of a method for inducing a hypoxic bolus in the subject.

FIG. 3 is a graph showing a dissociation curve for oxyhemoglobin.

FIG. 4 is an illustration of a user interface during exemplary performance of the method of FIG. 2.

FIG. 5 is another illustration of a user interface during exemplary performance of the method of FIG. 2.

FIG. 6 is a graph showing experimental results during exemplary performance of the method of FIG. 2.

FIG. 7A is a graph showing lung volume during exemplary performance of the method of FIG. 2.

FIG. 7B is another graph showing lung volume during exemplary performance of the method of FIG. 2.

FIG. 7C is another graph showing lung volume during exemplary performance of the method of FIG. 2.

FIG. 7D is another graph showing lung volume during exemplary performance of the method of FIG. 2.

FIG. 7E is another graph showing lung volume during exemplary performance of the method of FIG. 2.

FIG. 7F is another graph showing lung volume during exemplary performance of the method of FIG. 2.

### DETAILED DESCRIPTION

### List of Acronyms

“AT” herein refers to arrival time.
“AIF” herein refers to arterial input function.
“CBV” herein refers to cerebral blood volume.
“CBF” herein refers to cerebral blood flow.
[dOHb] herein refers to the concentration of deoxyhemoglobin in a subject's blood.
“ERV” herein refers to expiratory reserve volume.
“FRC” herein refers to functional residual capacity.
“Gd” herein refers to gadolinium.
“IRV” herein refers to inspiratory reserve volume.
“MTT” herein refers to mean transit time.
“PₐO₂” herein refers to arterial partial pressure of oxygen.
“P_{ET}O₂” herein refers to end tidal partial pressure of oxygen in an exhaled gas.
“PO₂” herein refers to the partial pressure of oxygen in a gas, especially an inhaled gas.
“PCO₂” herein refers to the partial pressure of carbon dioxide in a gas, especially an inhaled gas.
“RV” herein refers to residual volume.
“SaO₂” herein refers to arterial blood-oxygen saturation.
“SGD” herein refers to sequential gas delivery and may refer either to a device or a method.
“TTP” herein refers to time to peak.
“[dOHb]” is the concentration of deoxyhemoglobin in the blood (see SaO₂).
“SaO₂” is the saturation of hemoglobin with oxygen as a percent of total hemoglobin. The total hemoglobin concentration in blood×(100−SaO₂)=[dOHb].
“VT” herein refers to tidal volume.

According to one method of using deoxyhemoglobin as a contrast agent, a baseline level of blood oxygenation is established, an abrupt change is implemented to reduce the lung PO₂ to a lower PO₂ target level, followed by re-oxygenation to the baseline level. These PO₂ changes are followed by changes in arterial [dOHb] they implement. The changes in BOLD signal caused by the changes in [dOHb] are the arterial input function (AIF).

According to one method of using deoxyhemoglobin as a contrast agent, a hypoxic baseline is established in the subject and a rapid increase in hemoglobin saturation is implemented by restoring normal lung PO₂, thereby optimizing the arterial input function (AIF).

A drawback to known methods is the extended period of time required to reach the hypoxic baseline. The more rapidly the hypoxic baseline is reached, the shorter the duration of hypoxia and of the measurement. When target hypoxia is reached, re-oxygenation can be implemented. The duration of hypoxia is then minimized when the target level of hypoxia is reached in a square wave function. The issue identified by the present disclosure is that it is technically very challenging to implement a step change to hypoxia from a near normoxic baseline that has the same profile as the normoxic transition from a hypoxic baseline.

The present disclosure provides a method and system for controlling the reduction of partial pressure of oxygen in the lungs of a subject to produce transient hypoxic steps, which in turn result in transient decreases in hemoglobin oxygen saturation, which in turn are passed on to the arteries and the tissues such as the brain. Such transient hypoxic steps may be safe, insensible to the subject, too brief to affect cerebral blood flow, and repeatable with relatively high precision if required for monitoring of hemodynamic parameters.

The above aspects can be attained by optimizing the speed and range of the transition to low PO₂ in the lung such that it approaches the PO₂ and timing profile of reoxygenation from a hypoxic profile. However, such hypoxic step changes are much more difficult to implement than during re-oxygenation. The solution provided by the present disclosure is to control the independent variables which contribute to the rate of change in partial pressure of oxygen (PO₂) in the lung. These variables may be controlled in alone or in aggregate to generate an hypoxic transition from normoxia that is comparable to that generated from reoxygenation following a hypoxic baseline. In particular, the resulting rapid increase in [dOHb] can be implemented by breathing more deeply, on exhalation, exhaling past at least a portion of the resting level functional residual capacity, lowering the PO₂ at baseline, increasing the breathing rate above resting levels, increasing the targeted PO₂, and increasing the partial pressure of carbon dioxide (PCO₂) in the lung to shift the oxygen dissociation curve to the right.

These variables can be controlled using a sequential gas delivery device. A sequential gas delivery device can model the oxygen absorbed by the blood arriving in the pulmonary artery (which depends on cardiac output) and controls the amount of oxygen in the subsequent breath (which depends on the size of the breath and the concentration of oxygen).

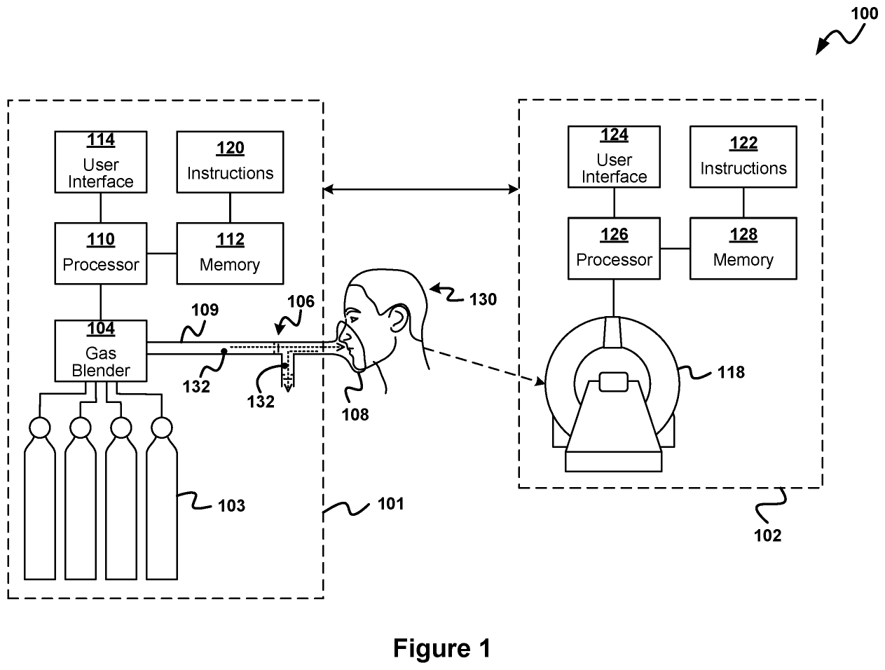

FIG. 1 shows a system 100 for controlling a rate of change in the [dOHb] in a subject. The system 100 includes a device 101 to provide sequential gas delivery to a subject 130 and target a PₐO₂ while maintaining normocapnia. The system 100 further includes a magnetic resonance imaging (MRI) system 102. The device 101 includes gas supplies 103, a gas blender 104, a mask 108, a processor 110, memory 112, and a user interface device 114. The device 101 may be configured to control end-tidal partial pressure of CO₂ (P_{ET}CO₂) and end-tidal partial pressure of O₂ (P_{ET}O₂) by generating predictions of gas flows to actuate target end-tidal values. The device 101 may be an RespirAct™ device (Thornhill Medical™: Toronto, Canada) specifically configured to implement the techniques discussed herein. For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 103 may provide carbon dioxide, oxygen, nitrogen, and air, for example, at controllable rates, as defined by the processor 110. A non-limiting example of the gas mixtures provided in the gas supplies 103 is:
a. Gas A: 10% O₂, 90% N₂;
b. Gas B: 10% O₂, 90% CO₂;
c. Gas C: 100% O₂; and
d. Calibration gas: 10% O₂, 9% CO₂, 81% N₂.

The gas blender 104 is connected to the gas supplies 103, receives gases from the gas supplies 103, and blends received gases as controlled by the processor 110 to obtain a gas mixture, such as a first gas (G1) and a second gas (G2) for sequential gas delivery.

The second gas (G2) is a neutral gas in the sense that it has about the same PCO₂ as the gas exhaled by the subject 130, which includes about 4% to 5% carbon dioxide. In some examples, the second gas (G2) may include gas actually exhaled by the subject 130. The first gas (G1) has a composition of oxygen that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 110 may control the gas blender 104, such as by electronic valves, to deliver the gas mixture in a controlled manner.

The mask 108 is connected to the gas blender 104 and delivers gas to the subject 130. The mask 108 may be sealed to the subject's face to ensure that the subject only inhales gas provided by the gas blender 104 to the mask 108. In some examples, the mask is sealed to the subject's face with skin tape such as Tegaderm™ (3M™, Saint Paul, Minnesota). A valve arrangement 106 may be provided to the device 101 to limit the subject's inhalation to gas provided by the gas blender 104 and limit exhalation to the room. In the example shown, the valve arrangement 106 includes an inspiratory one-way valve from the gas blender 104 to the mask 108, a branch between the inspiratory one-way valve and the mask 108, and an expiratory one-way valve at the branch. Hence, the subject 130 inhales gas from the gas blender 104 and exhales gas to the room.

The gas supplies 103, gas blender 104, and mask 108 may be physically connectable by a conduit 109, such as tubing, to convey gas. Any number of sensors 132 may be positioned at the gas blender 104, mask 108, and/or conduits 109 to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor may be connected to and cooperate with the memory 112 that stores instructions and data.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface device 114 may include a display device, touchscreen, keyboard, buttons, the like, or a combination thereof to allow for operator input and/or output.

Instructions 120 may be provided to carry out the functionality and methods described herein. The instructions 120 may be directly executed, such as a binary file, and/or may include interpretable code, bytecode, source code, or similar instructions that may undergo additional processing to be executed. The instructions 120 may be stored in the memory 112.

System 100 further includes an MRI system 102 for conducting magnetic resonance imaging on the subject 130. A suitable MRI system may include an imaging device such as a 3T MRI system (Signa HDxt-GE Healthcare, Milwaukee). The MRI system 102 may further include a processor 126, memory 128, and a user interface 124. Any description of the processor 126 may apply to the processor 110 and vice versa. Likewise, any description of memory 128 may apply to memory 112 and vice versa. Similarly, any description of instructions 112 may apply to instructions 120 and vice versa. Also, any description of user interface 124 may apply to user interface 114, and vice versa. In some implementations, the MRI system 102 and the device 101 share one or more of a memory, processer, user interface, and instructions, however, in the present disclosure, the MRI system 102 and the device 101 will be described as having respective processors, user interfaces, memories, and instructions. The processor 110 of the device 101 transmits data to the processor 126 of the MRI system 102. The system 100 may be configured to synchronize MRI imaging obtained by the MRI system 102 with measurements obtained by the device 101.

The processor 126 may retrieve operating instructions 122 from the memory or may receive operating instructions 122 from the user interface 124. The operating instructions 122 may include image acquisition parameters. The parameters may include an interleaved echo-planar acquisition consisting of a number of contiguous slices, a defined isotropic resolution, a diameter for the field of view, a repetition time, and an echo time. In one implementation, the number of contiguous slices is 27, the isotropic resolution is 3 mm, the field of view is 19.6 cm, the echo time is 30 ms, and the repetition time (TR) is 2000 ms, however a range of values will be apparent to a person of ordinary skill in the art. The operating instructions 122 may also include parameters for a high-resolution T1-weighted SPGR (Spoiled Gradient Recalled) sequence for co-registering the BOLD images and localizing the arterial and venous components. The SPGR parameters may include a number of slices, a dimension for the partitions, an in-plane voxel size, a diameter for the field of view, an echo time, and a repetition time. In one implementation, the number of slices is 176 m, the partitions are 1 mm thick, the in-plane voxel size is 0.85 by 0.85 mm, the field of view is 22 cm, the echo time is 3.06 ms, and the repetition time (TR) is 7.88 ms.

The processor 126 may be configured to analyze the images using image analysis software such as Matlab 2015a and AFNI or other processes generally known in the art. As part of the analysis, the processor 126 may be configured to perform slice time correction for alignment to the same temporal origin and volume spatial re-registration to correct for head motion during acquisition. The processor 126 may be further configured to perform standard polynomial detrending. In one implementation, the processor 126 is configured to detrend using AFNI software 3dDeconvolve to obtain detrended data.

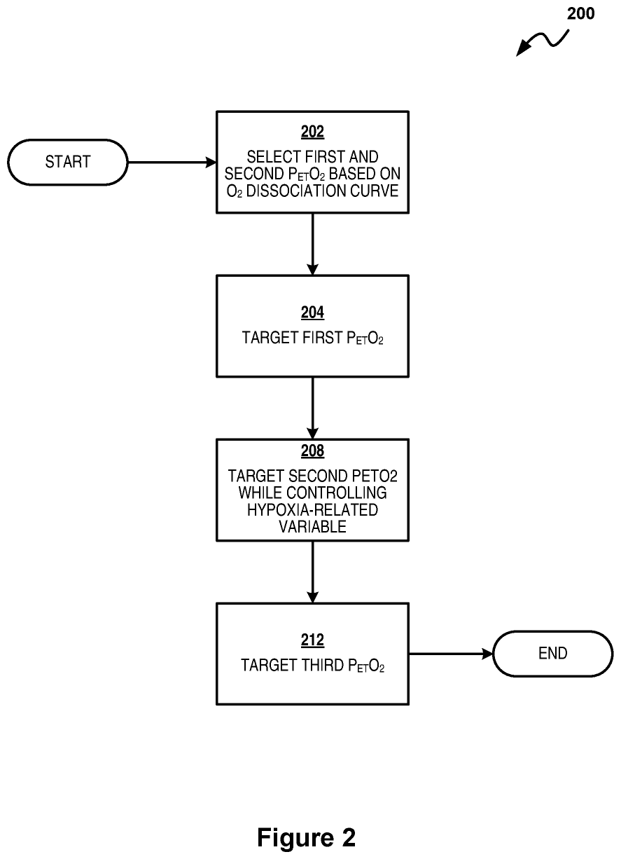

FIG. 2 shows an example method 200 of controlling the rate of change in the concentration of deoxyhemoglobin in the subject. In particular, the device 101 may be programmed to implement a rapid decrease in [dOHb] from a normoxic baseline. The method 200 may be implemented by instructions 120 stored on memory 112 and implemented by processor 110 and/or instructions 122 stored in memory 128 and implemented by processor 126.

At block 202, instructions 120 control the device 101 to select a first P_{ET}O₂ and a second P_{ET}O₂ based on a target [dOHb] with the relationship between P_{ET}O₂ and hemoglobin saturation is described by the oxygen-hemoglobin dissociation curve.

The first P_{ET}O₂ is selected to establish a baseline for the hypoxic step and the “first P_{ET}O₂” may be used interchangeably with the “baseline P_{ET}O₂”. The first P_{ET}O₂ may be selected to maintain PₐO₂ at or near normoxia. In some examples, the first P_{ET}O₂ is between approximately 80 mmHg and approximately 100 mmHg. In particular examples, the first P_{ET}O₂ is approximately 80 mmHg. The second P_{ET}O₂ is selected to induce a measurable [dOHb] signal in the subject and the “second P_{ET}O₂” may be used interchangeably with the “target P_{ET}O₂”. The second P_{ET}O₂ is lower than the first P_{ET}O₂ and may be selected to induce hypoxia in the subject. In some examples, the second P_{ET}O₂ is approximately 40 mmHg.

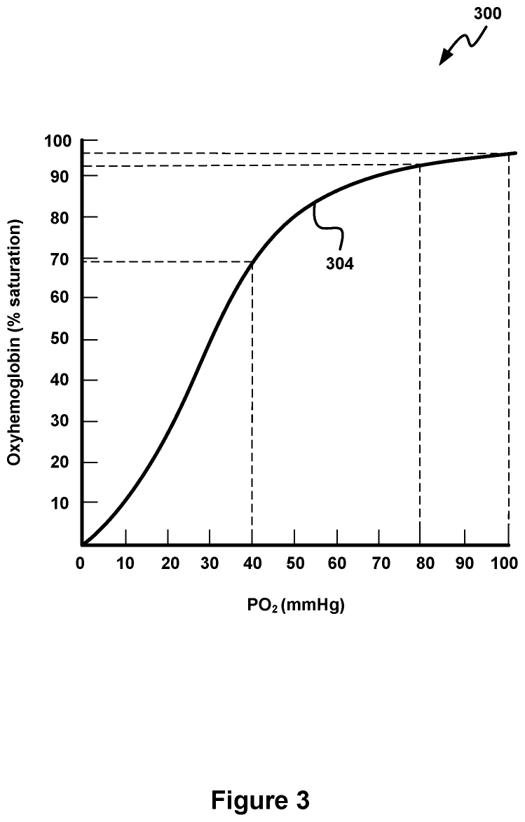

The first and second P_{ET}O₂ are selected according to target [dOHb] with the relationship of P_{ET}O₂ and [dOHb] described in the oxygen-hemoglobin dissociation curve. An example of an oxygen-hemoglobin dissociation curve is shown in FIG. 3 at 300. FIG. 300 shows a graph with the oxyhemoglobin (measured in % saturation) plotted on the y-axis and PO₂ (measured in mmHg) plotted on the x-axis. Under normal physiologic conditions, the dissociation curve 304 is fairly flat when PO₂ is above 80 mmHg, so fluctuations to PO₂ in this range will minimally affect the BOLD signal. This is illustrated by the dotted lines which show that decreasing the PO₂ from 100 mmHg to 80 mmHg only minimally reduces the oxyhemoglobin as compared to a decrease from 80 mmHg to 40 mmHg. To optimize the hypoxic signal, the first and second P_{ET}O₂ may be selected to include a portion of the curve 304 with a greater slope.

To focus on a portion of the curve with a larger slope, the first P_{ET}O₂ may be selected to be lower than normoxia. At rest, P_{ET}O₂ is typically above 100 mmHg, but values as low as about 80 mmHg are well tolerated by most subjects for extended periods of time. Therefore, the first P_{ET}O₂ may be selected between approximately 80 mmHg and approximately 100 mmHg. The minimum required duration of baseline to implement this method is as short as 1 breath. The second P_{ET}O₂ at target hypoxia may vary with subject tolerance and requirement to enable the contrast function of [dOHb]. Generally, it will be desirable to select the lowest PO₂ that the subject can tolerate for the time required to establish a baseline P_{ET}O₂.

The difference between the first and second P_{ET}O₂ should be sufficiently large to elicit a change in [dOHb] that causes a measurable magnetic signal, however the smaller the difference between the first and second P_{ET}O₂, the faster and more accurately the target can be achieved. Therefore, the second P_{ET}O₂ may be elevated as much as the permitted by the signal-noise ratio. In some examples, the second P_{ET}O₂ is approximately 30 mmHg. In other examples, the second P_{ET}O₂ is approximately 40 mmHg. In further examples, the second P_{ET}O₂ is approximately 50 mmHg. In yet further examples, the second P_{ET}O₂ is approximately 60 mmHg.

In a non-limiting example, the first P_{ET}O₂ is approximately 80 mmHg and the second P_{ET}O₂ is approximately 40 mmHg. An a further non-limiting example, the first P_{ET}O₂ is approximately 100 mmHg and the second P_{ET}O₂ is approximately 40 mmHg.

At block 204, instructions 120 control the device 101 to target the first P_{ET}O₂ in the subject.

As part of block 204, the instructions 120 control the sensor 132 to measure the subject's actual P_{ET}O₂. Once the first P_{ET}O₂ is reached, the instructions 130 may proceed to implement block 208. In some examples, the instructions 130 only proceed to block 208 once the first P_{ET}O₂ has been maintained for a pre-determined duration of time or a pre-determined number of breaths.

As part of block 204, the device 102 may measure a first magnetic signal in a voxel of the subject's brain. In one example, the device 102 measures a T2* dependent signal, also called the Blood Oxygen-Level Dependent (BOLD) signal. To ensure that the measurement corresponds with the targeted P_{ET}O₂, the device 102 may measure the first magnetic signal only after the processor 110 determines that the first P_{ET}O₂ has been reached. The first magnetic signal may be stored in the memory 128.

At block 208, the instructions 120 control the device 101 to target the second P_{ET}O₂ in the subject.

While the device 101 is targeting the second P_{ET}O₂, one or more variables that contribute to the rate of change of PO₂ are controlled. The one or more variables include a breathing pattern or PCO₂, or a combination thereof.

Breathing deeply allows the device 101 to decrease P_{ET}O₂ more quickly. A number of patterns of deep breathing are contemplated. In some embodiments, deep breathing is characterized by a tidal volume that is greater than the subject's resting tidal volume. In particular examples, the tidal volume is between the subject's resting tidal volume and the subject's vital capacity. In other embodiments, deep breathing is characterized by exhaling until the subject's lung volume is less than the subject's functional residual capacity. In other words, the subject exhales a portion of their normal expiratory reserve volume (ERV) causing the lung volume to approach the residual volume (RV). By exhaling a portion of the ERV, the subject reduces the FRC which allows the device 101 to more quickly dilute the oxygen in the FRC.

The rate at which the P_{ET}O₂ decreases may be further accelerated if the subject breathes more rapidly. While the device 101 is targeting the second P_{ET}O₂, the subject may be breathing at a rate faster than the subject's resting breath rate. In one example, the subject breathes every 4 seconds. In a further example, the subject breathes every 3 seconds. In yet a further example, the subject breathes every 2 seconds.

Block 208 may be performed while the subject is implementing one or more of the above-described breathing patterns. In some examples, block 208 is performed while the subject is implementing all of the above-described breathing patterns. In other words, the tidal volume is greater that the subject's resting tidal volume, the subject exhales a portion of the subject's ERV, and the subject is breathing at a rate faster than the subject's resting breath rate.

To guide the breathing pattern, instructions may be given to the subject. The instructions may be delivered by a technician or medical professional or the instructions may be displayed on the user interface 114. The instructions may be delivered to the subject prior to performing method 200 or during the performance of method 200. In a non-limiting example, the breathing instructions comprise: breathe all the way out, take a deep breath, and repeat 7 more times when prompted.

The dissociation curve 304 may be further optimized by manipulating physiological conditions in the subject. The curve 304 can be shifted to the right by decreasing pH, increasing 2,3-diphosphoglycerate (2,3-DPG), or increasing temperature. In one example, the device 101 increases the PCO₂ in the subject above a resting PCO₂. Since pH is inversely proportional to PCO₂, increasing the PCO₂ will decrease pH and shift the curve 304 to the right. The shift increases the [dOHb] in the subject for a given arterial PO₂. The right shift allows the device 101 to implement greater changes to [dOHb] at a higher range of PO₂. In some examples, the device 101 maintains an elevated PCO₂ in the subject throughout performance of the method 200. In other examples, the device 101 only increases the PCO₂ while targeting the second P_{ET}O₂ at block 208.

As part of block 208, the instructions 120 control the sensor 132 to measure the subject's actual P_{ET}O₂. Once, the second P_{ET}O₂ is reached, the instructions 130 may proceed to implement block 208. In some examples, the instructions 130 only proceed to block 212 once the second P_{ET}O₂ has been maintained for a pre-determined duration of time or a pre-determined number of breaths. In further examples, the instructions 130 proceed to block 212 once the subject has inhaled a cumulative volume of gas approximately equal to 3 times the subject's functional residual capacity.

As part of block 208, the device 102 may measure a second magnetic signal in the voxel. In one example, the device 102 measures the BOLD signal. To ensure that the measurement corresponds with the targeted P_{ET}O₂, the device 102 may measure the second magnetic signal only after the processor 110 determines that the second P_{ET}O₂ has been reached. The second magnetic signal may be stored in the memory 128.

At block 212, the SGD device 101 targets a third P_{ET}O₂. The third P_{ET}O₂ is selected to return the subject to a state at or near normoxia so that the hypoxic state was merely transient. Therefore, the third P_{ET}O₂ is higher than the second P_{ET}O₂, but is not necessarily equal to the first P_{ET}O₂. In some examples, the third P_{ET}O₂ is between approximately 80 mmHg and approximately 100 mmHg. In particular examples, the first P_{ET}O₂ is approximately 80 mmHg.

Where the PO₂ has been reduced, a single breath can restore full hemoglobin saturation using dilution kinetics. Such kinetics are demonstrated in Poublanc et al. 2021 MRM 2021, which is incorporated herein by reference. The gas of a single breath is thoroughly dispersed in the lung, simultaneously oxygenating the blood in each healthy alveolus. It then passes via the pulmonary vein through the left atrium, left ventricle and into the arteries with minimal dispersion. On arrival of the fully oxygenated blood to the brain, the [dOHb] abruptly decreases to zero, resulting in the BOLD signal.

As part of block 212, the instructions 120 control the sensor 132 to measure the subject's actual P_{ET}O₂. Once, the third P_{ET}O₂ is reached, the instructions 130 proceed to implement block 208. In some examples, the instructions 130 only proceed to block 208 once the first P_{ET}O₂ has been maintained for a pre-determined duration of time or a pre-determined number of breaths.

As part of block 212, the device 102 may measure a third magnetic signal in a voxel of the subject's brain. In one example, the device 102 measures the BOLD signal. To ensure that the measurement corresponds with the targeted P_{ET}O₂, the device 102 may measure the third magnetic signal only after the processor 110 determines that the third P_{ET}O₂ has been reached. The third magnetic signal may be stored in the memory 128.

As part of the method 200, the processor 120 may compute a perfusion metric based on the first and second magnetic signals.

As described above, several factors may be controlled to more quickly achieve the hypoxic second P_{ET}O₂: the first P_{ET}O₂ may be depressed and the second P_{ET}O₂ may be raised to align with a steeper segment of the oxygen-hemoglobin dissociation curve; PCO₂ may be increased to shift the dissociation curve to the right; the breathing rate may be increased; the tidal volume may be increased; and the functional residual capacity may be reduced. The method 200 may comprise controlling one or more of the above factors. In some examples, all of the above factors are controlled to institute a sharper transient hypoxic step.

By optimizing the kinetics of deoxygenation in the lung, the system and method effect dispersion of [dOHb] that is comparable to that obtained with re-oxygenation of [dOHb]. By employing the system and method, a rapid, transient hypoxic signal may be induced for the purposes of perfusion MRI.

The advantages of method 200 may be better understood in reference to dilution kinetics and wash-out kinetics.

### Dilution Kinetics

The concentration of gas in the lung may be altered similarly to the use of concentrated dye to change the color of water in a container. For water, the following calculation may be used: required volume of dye=desired concentration of dye in the water×(volume of water in the glass+volume of dye)−amount of dye already in the water. For oxygen in the lungs, the dilution can be modelled according to Formula 1:

$$Volume of inhaled {O}_{2} = desired {O}_{2} concentration \times ( FRC + VT ) - {O}_{2} in lung \qquad Formula 1$$

The concentration of oxygen in the lung may be increased to a target level by calculating (i) the functional residual capacity (FRC), (ii) the volume of oxygen already in the FRC; and (iii) the concentration of the inspired oxygen. The concentration of oxygen may then be targeted by limiting the volume of oxygen to that resulting in the net lung oxygen concentration. This capability is available only in certain sequential gas delivery devices such as the RespirAct™ (Thornhill Medical™; Toronto, Canada). As oxygen in the lung is about 20% of gas, and oxygen is available at 100%, a large range of increases in lung PO₂ can occur with a single breath, depending on its volume and the volume of the FRC. If the FRC is 3000 ml, and has little oxygen, say at PO₂ of 40 mmHg, an unambitious target PO₂ of 100 mmHg may be attained within one or two breaths by dilution kinetics.

Similar considerations apply for decreasing oxygen concentration in the lung. The difference is that the oxygen in the FRC is diluted. This requirement means that even if the inhaled gas has no oxygen, such as pure nitrogen, the dilution is very difficult. For example, reducing the oxygen concentration at FRC by half, one would need to inhale a volume of nitrogen equal to the FRC. In a typical adult male, the FRC is about 3000 ml and a breath that size is difficult to accomplish for most people. At the end of a typical exhalation when breathing room air, the oxygen concentration in the FRC is about 100 mmHg. Targeting a PO₂ of 40 mmHg requires more than dilution by half. If done with sequential breaths, each additional breath/dilution is progressively inefficient in lowering the PO₂ in the FRC. This may be an impediment to implementing an immediate reduction of lung oxygen concentration for generating an arterial input function for MRI testing.

### Wash-Out Kinetics.

While breathing, changing the inhaled gas to one with new concentration of gases, for example from room air (20% O₂; 80% N₂) to 4% oxygen, with the balance being N₂, the lung oxygen concentration decreases in a predictable manner with sequential breaths. When the cumulative volume of inhaled gas is equal to the FRC, the new gas makes up about 66% of the FRC. It is not 100% because with each exhalation, some of the previously inhaled nitrogen is exhaled. The closer the gas concentration in the lungs gets to the 4% nitrogen, the smaller is the absolute decrement of lung concentration of oxygen per breath, as is the property of a decreasing exponential approaching an asymptote. When a cumulative volume equal to three times FRC is inhaled, the lung is only 95% of the way to a new equilibrium lung oxygen concentration. In this example, breathing a cumulative volume equal to the FRC of the new gas is considered one time constant (τ).

The tissues of the body are continuously absorbing oxygen. For each circulation time, the PO₂ in the blood returning to the lungs is lower. The circulation time varies depending on each part of the body from about one second in the heart, to more than 25 seconds in the toes. If a net recirculation time of 15 seconds is assumed, this may be too long to contribute to a rapid reduction of PO₂ in, say, less than 5 seconds.

### A Multimodal Approach

The present disclosure shows that a substantial reduction of PO₂ in the lungs, such as reducing the PO₂ from 100 mmHg to 40 mmHg, can be accomplished by optimizing washout kinetics. Strategies to optimize the washout kinetics may require information regarding: the subject's FRC and the first P_{ET}O₂. Additionally, optimizing washout kinetics may require controlling the PO₂ of gas to be inhaled, the volume of the gas to be inhaled (i.e., the size of the breath) before the breath is executed, implementing a breathing pattern where exhalation continues below FRC towards the reserve volume (RV), and exploiting knowledge of the sigmoidal shape of the oxyhemoglobin dissociation curve which is flat at its upper end. Recall that the MRI signal depends on the concentration of [dOHb]—this means that linear reductions in baseline PO₂ from room air reduces the volume of oxygen in the FRC that needs to be diluted, but has little effect on oxygenation saturation, and therefore little effect on the BOLD signal.

This method combines the known approaches to lowering lung PO₂ such as taking larger breaths, with novel approaches to enhance the technique:
exhaling below the FRC towards RV
reducing the PO₂ at high baseline
taking large breaths
taking more frequent breaths
increasing the second PO₂ as signal-to-noise ratio (SNR) allows

Method 200 further optimizes wash-out kinetics with sequential gas delivery. A selected PO₂ level can be targeted if the inhaled volume of a hypoxic diluent is known. This system enables (i) the delivery of a known volume of diluent gas independent of tidal volume, and frequency of breathing, and (ii) the ability to separately target blood PO₂ and PCO₂. Both of these elements may enable the use of [dOHb] as a contrast agent.

The method 200 will now be explained by way of example.

### Example 1

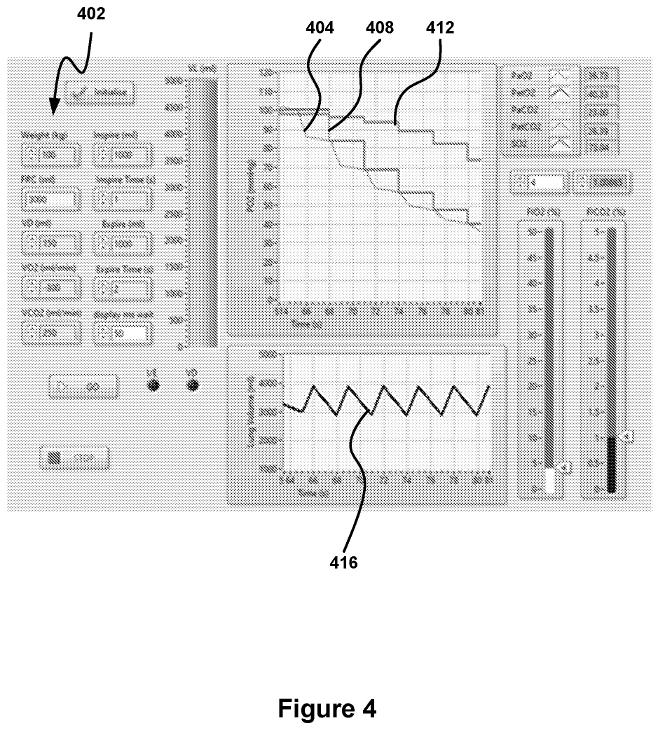

FIG. 4 shows exemplary performance of method 200 using system 100.

FIG. 4 illustrates the user interface 114 displaying the parameters implemented by instructions 120 and the resulting PO₂ detected by the sensor 132. As indicated generally at 402, the instructions implemented a FRC of 3000 ml, a tidal volume (V_{T}) of 1000 ml above FRC, and a breathing rate of 3 seconds per breath. The arterial partial pressure of oxygen (PₐO₂) is shown at 404, the measured P_{ET}O₂ is shown at 408, the arterial blood-oxygen saturation (SaO₂) is shown at 412, and lung volume is shown at 416. The measured P_{ET}O₂ 408 shows that the system 100 targeted a first P_{ET}O₂ of 100 mmHg and then targeted a second P_{ET}O₂ of 40 mmHg. The second P_{ET}O₂ was reached within 12 seconds and 5 breaths.

### Example 2

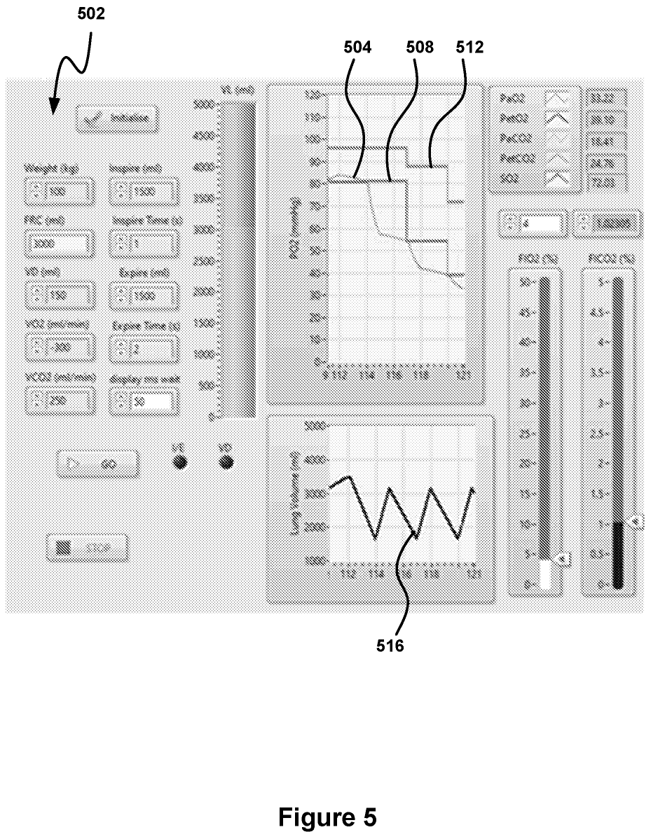

FIG. 5 shows another exemplary performance of method 200. FIG. 5 illustrates the user interface 114 displaying the parameters implemented by instructions 120 and the resulting PO₂ detected by the sensor 132. As indicated generally at 502, the instructions implemented a FRC of 3000 ml, a tidal volume (V_{T}) of 1500 ml above FRC, and a breathing rate of 3 seconds per breath. The arterial partial pressure of oxygen (PₐO₂) is shown at 504, the measured P_{ET}O₂ is shown at 508, the arterial blood-oxygen saturation (SaO₂) is shown at 512, and lung volume is shown at 516. The measured P_{ET}O₂ 508 shows that the system 100 targeted a first P_{ET}O₂ of 80 mmHg and then targeted a second P_{ET}O₂ of 40 mmHg. The second P_{ET}O₂ was reached within 5 seconds and 2 breaths.

### Example 3

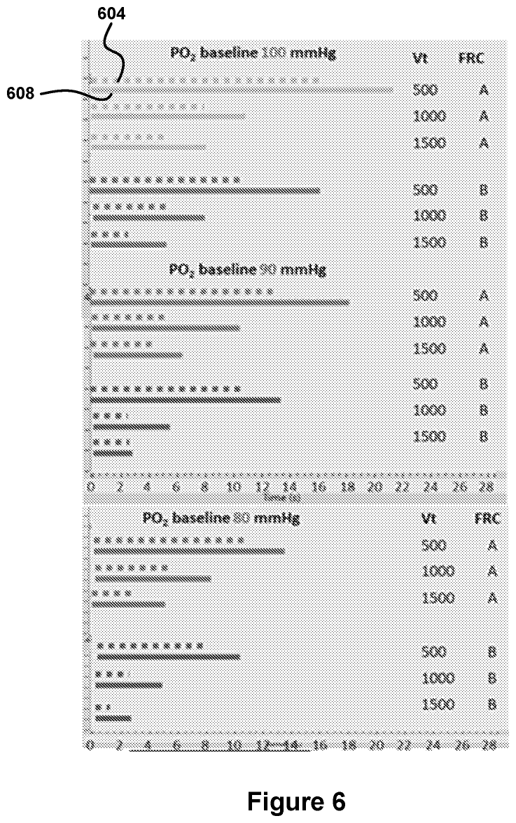

FIG. 6 shows another exemplary performance of method 200. FIG. 6 illustrates the user interface 114 displaying the variables and time required to reach a second P_{ET}O₂ of 40 mmHg. The broken lines 604 indicate the number of breaths required to reach the second P_{ET}O₂. The solid lines 608 indicate the time required to reach the second P_{ET}O₂, as measured in seconds on the x-axis. Vₜ indicates the tidal volume. FRC indicates a breathing pattern of either A or B. Breathing pattern A is characterized by the subject inhales above the FRC. Therefore, when the tidal volume is the subject's resting tidal volume, the subject is breathing normally. When the tidal volume is greater than the subject's resting tidal volume, the subject exhales at least a portion of the IRV. Breathing pattern B is characterized by the subject only exhaling below the FRC, thereby exhaling at least a portion of the ERV.

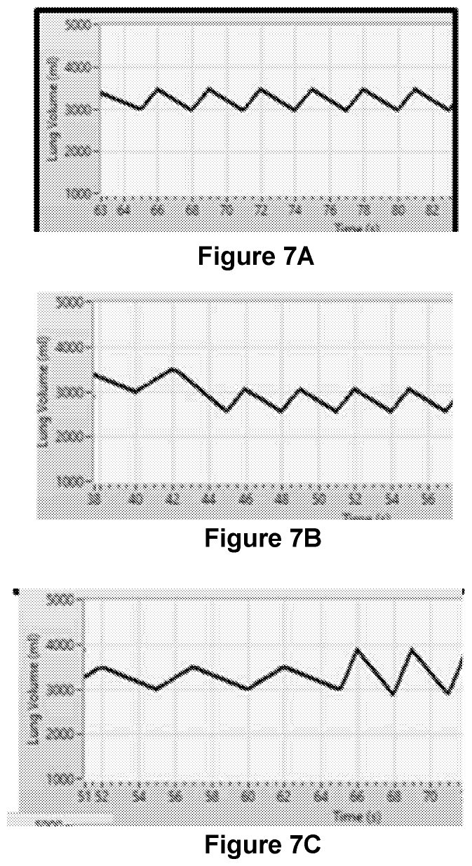

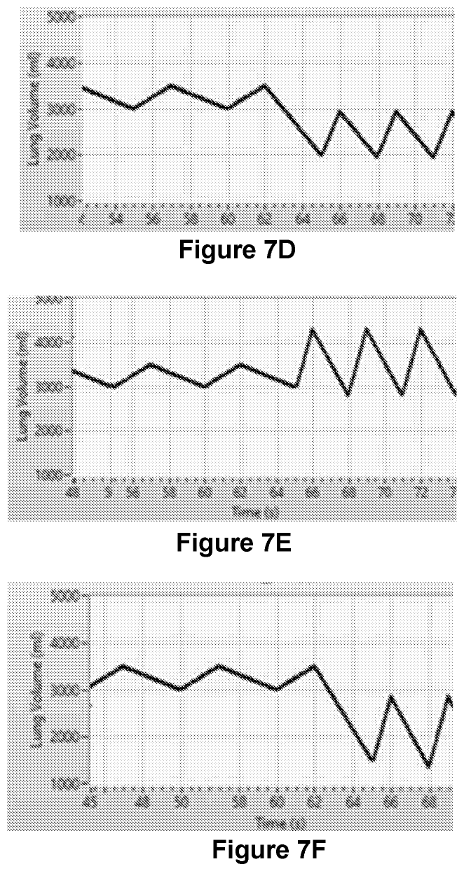

FIGS. 7A to 7F show a series of line graphs comparing breathing pattern A to breathing pattern B. In each graph, the lung volume (ml) is plotted on the y-axis and time (seconds) is plotted on the x-axis. FIG. 7A shows breathing pattern A with a tidal volume of 500 mL. FIG. 7B shows breathing pattern B with a tidal volume of 500 mL. FIG. 7C shows breathing pattern A with a tidal volume of 1000 mL. FIG. 7D shows breathing pattern B with a tidal volume of 1000 mL. FIG. 7E shows breathing pattern A with a tidal volume of 1500 mL. FIG. 7F shows breathing pattern B with a tidal volume of 1500 mL.

FIG. 6 demonstrates that each breathing mode is independently capable of shortening the time to target the second P_{ET}O₂. Increasing the tidal volume (Vₜ) above the resting tidal volume reduces the time required to reach the second P_{ET}O₂. Breathing below the FRC (as in breathing pattern B) instead of above the FRC (as in breathing pattern A) reduces the time required to reach the second P_{ET}O₂. Lowering the first (baseline) P_{ET}O₂ reduces the time required to reach the second P_{ET}O₂. Furthermore, FIG. 6 shows that combining strategies at least has an additive effect on the time required to reach the second P_{ET}O₂. A P_{ET}O₂ of 40 mmHg can be implemented in less than 2 breaths using breathing pattern B, a tidal volume of 1500 mL, and a first (baseline) P_{ET}O₂ of 80 mmHg. This is a substantial improvement over the 21 breaths required to reach a P_{ET}O₂ of 40 mmHg using breathing pattern A, a tidal volume of 500 mL, and a first (baseline) P_{ET}O₂ of 100 mmHg.

It should be recognized that features and aspects of the various examples provided above can be combined into further examples that also fall within the scope of the present disclosure. In addition, the figures are not to scale and may have size and shape exaggerated for illustrative purposes.

What is claimed is:

1. A method of inducing a deoxyhemoglobin bolus in a subject, the method comprising:
selecting a first end-tidal partial pressure of oxygen (P_{ET}O₂) and a second P_{ET}O₂ based on target [dOHb] where the relationship between P_{ET}O₂ and [dOHb] is described by an oxygen-hemoglobin dissociation curve, the second P_{ET}O₂ lower than the first P_{ET}O₂;
targeting the first P_{ET}O₂ in the subject using a sequential gas delivery device;
after targeting the first P_{ET}O₂, targeting the second P_{ET}O₂ using the sequential gas delivery device while controlling a variable that contributes to the rate of change of the partial pressure of oxygen (PO₂) in the subject's lung; and
after targeting the second P_{ET}O₂, targeting a third P_{ET}O₂ using the sequential gas delivery device, the third P_{ET}O₂ higher than the second P_{ET}O₂.

2. The method of claim 1 wherein the variable comprises a breathing pattern, and wherein the subject inhales a tidal volume greater than the subject's resting tidal volume while the second P_{ET}O₂ is targeted.

3. The method of claim 2 wherein the tidal volume is between the subject's resting tidal volume and the subject's vital capacity.

4. The method of claim 1 wherein the variable comprises a breathing pattern, and wherein the subject exhales until the subject's lung volume is less than the subject's functional residual capacity (FRC) at rest, while the second P_{ET}O₂ is targeted.

5. The method of claim 1 wherein the variable comprises a breath rate, and wherein the subject breathes at a rate faster than the subject's resting breath rate while the second P_{ET}O₂ is targeted.

6. The method of claim 1 wherein the variable is PCO₂, the method further comprising targeting a PCO₂ in the subject using the sequential gas delivery device while targeting the second P_{ET}O₂, the PCO₂ selected to shift the oxyhemoglobin dissociation curve to the right and increase the [dOHb] in the subject for a given arterial PO₂.

7. The method of claim 1 wherein selecting the first P_{ET}O₂ and second P_{ET}O₂ based on the oxygen-hemoglobin dissociation curve comprises determining the slope of the oxygen-hemoglobin dissociation curve and selecting the first P_{ET}O₂ and second P_{ET}O₂ based on the slope of the oxygen-hemoglobin dissociation curve.

8. The method of claim 7 wherein the first P_{ET}O₂ is between 80 and 100 mmHg

9. The method of claim 8 wherein the first P_{ET}O₂ is approximately 80 mmHg.

10. The method of claim 7 wherein the second P_{ET}O₂ is approximately 40 mmHg.

11. The method of claim 1 further comprising targeting the second P_{ET}O₂ until the subject inhales a cumulative volume of gas approximately equal to 3 times the subject's FRC.
